(19) United States
(12) Patent Application Publication
(10) Pub. No.: US 2022/0047287 A1
(43) Pub. Date: Feb. 17, 2022

(54) INTERCHANGEABLE PROBE TIPS FOR CALCULI FRACTURE AND REMOVAL

(71) Applicant: GYRUS ACMI, INC. D/B/A OLYMPUS SURGICAL TECHNOLOGIES AMERICA, Westborough, MA (US)

(72) Inventors: Eric Stender, Champlin, MN (US); Charles Baker, Rogers, MN (US); Thomas J. Holman, Princeton, MN (US); Arthur J. Bertelson, Buffalo, MN (US)

(21) Appl. No.: 17/398,517

(22) Filed: Aug. 10, 2021

Related U.S. Application Data
(60) Provisional application No. 63/065,845, filed on Aug. 14, 2020.

(51) Int. Cl. A61B 17/22

(52) U.S. Cl. CPC A61B 17/22012; A61B 2017/0088

(57) ABSTRACT

A device for acoustic calculi fracture can include an acoustically-transmissive elongated probe body extending between a distal portion and a proximal portion and an acoustically-transmissive probe tip that can be selectively user-interchangeable with the probe body. The probe body can have a lumen longitudinally therethrough. A method of fracturing calculi can include interchanging a probe tip with a probe body by a user without requiring an additional tool and transmitting acoustic energy via the probe body and the probe tip to a calculus to at least partially fracture the calculus.

### CROSS-REFERENCE TO RELATED APPLICATION

This application claims the benefit of priority to U.S. Provisional Patent Application Ser. No. 63/065,845, filed Aug. 14, 2020, the contents of which are incorporated herein by reference in their entirety.

### TECHNICAL FIELD

The present document relates to techniques for breaking obstructions, such as physiological calculi or “stones” using lithotripsy, and more particularly to techniques for breaking the obstructions such as using laser lithotripsy.

### BACKGROUND

Medical endoscopes were first developed in the early 1800s and have been used to inspect inside the body. A typical endoscope has a distal end comprising an optical or electronic imaging system and a proximal end with controls such as for manipulating the device or for viewing the image. An elongate shaft connects the proximal and distal ends. Some endoscopes allow a physician to pass a tool down one or more working channels, for example, to resect tissue or retrieve objects.

Over the past several decades, several advances have been made in the field of endoscopy, and in particular relating to the breaking up of physiologic calculi in the bile ducts, urinary tract, kidneys, and gall bladder. Physiological calculi in these regions may block ducts and cause a patient a substantial amount of pain and therefore must be broken down and/or removed. Different techniques have been developed to break up stones, including ultrasonic or other acoustic lithotripsy, pneumatic lithotripsy, electro-hydraulic lithotripsy (EHL), and laser lithotripsy such as can include breaking up of calculi using a green light, YAG, or holmium laser.

### SUMMARY OF THE DISCLOSURE

In one approach to lithotripsy, a single probe with a non-detachable probe tip can be used in fragmenting and removing calculi. In this approach, a single type of probe tip, having a particular size and shape, is used for a variety of calculi, which may be of different hardness and size. Such an approach can limit flexibility for the operator to adjust the treatment of such calculi such as based on their shape and type with regards to the probe tip type.

The present disclosure provides, among other things, systems and methods of fragmenting or removing calculi with a device having an end-user interchangeable probe tip. Having a variety of different probe tip types and shapes can allow for the selection of a probe tip most suited to the particular procedure and calculi being treated. Giving the end-user a choice of a particular probe tip can allow for more efficient fragmentation or removal of such calculi. For example, a softer calculus can be fragmented easier when treated with a square-cut probe tip, while a harder calculus can be fragmented easier when treated with a more chiseled probe tip.

The interchangeable probe tip can be configured and manufactured based on an individual patient's needs or based on needs presented by a particular calculus. For example, a diagnostic tool can be used to identify the type and size of calculus needing fragmentation and/or removal, and the tip can be appropriately shaped and manufactured for fragmenting and/or removing that particular calculus. Such an interchangeable probe tip can also help extend the lifetime of the reusable probe body, so that an operator can continue to use the same probe body, thereby reducing treatment expense.

The interchangeable probe tip can allow for tailoring or optimization of calculus fragmentation and/or removal procedures, such as can help decreasing procedure time. Additionally, the interchangeable probe tip can potentially help lower operating costs by extending the working life of the probe as a whole such as by allowing the end-user to replace worn or undesired probe tips as needed.

In an example, a device for acoustic calculi fracture can include an acoustically-transmissive elongated probe body extending between a distal portion and a proximal portion. The probe body can include a lumen extending longitudinally through the probe body and one or more acoustically-transmissive probe tips that are selectively user-interchangeable with the probe body.

In an example, a kit for use with a calculi fracture device can include a plurality of different acoustically-transmissive probe tips that are selectively user-interchangeable with a probe body of a lithotripsy device, such as without requiring a separate tool.

In an example, a method of fracturing calculi can include an end-user selecting or interchanging a probe tip with a probe body, such as without requiring an additional tool, and transmitting acoustic energy via the probe body and the selected probe tip to a calculus to at least partially fracture the calculus.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic diagram of a calculi fracture and removal device with an interchangeable probe tip in an example.

FIGS. 2A-2B illustrate perspective views of an interchangeable probe tip in an example.

FIGS. 3A-3C illustrate schematic diagrams of an interchangeable probe tip in an example.

FIGS. 4 illustrates a schematic diagram of an interchangeable probe tip in an example.

FIG. 5 illustrates a schematic diagram of an interchangeable probe tip in an example.

FIG. 6 illustrates a schematic diagram of an interchangeable probe tip in an example.

FIG. 7 illustrates a flow chart showing a method of applying an interchangeable probe tip in an example.

### DETAILED DESCRIPTION

Calculi fracture and removal can include an end-user interchangeable probe tip (or kit of tips) such as can be selected or swapped out, such as depending on the type or characteristic (e.g., hardness) of a calculus (e.g., kidney stone) being treated. Tip features can include a fluid inlet hole or one or more axial grooves, for example. Different tips can have various morphologies or can include or be made of various materials, such as a ceramic or composite.

For an end-user operator acoustically fragmenting and removing calculi, reduced fragmentation time is desired. This can be helped by using a larger amplitude acoustic fragmentation signal, but such larger amplitudes can result in increased probe wear or premature probe failure. The operator can desire to reduce fragmentation time without probe failure, and to take less time overall to complete the procedure once the lithotripsy device is inserted into the patient, and before the puncture site is closed. Additionally, it can be desired that no residual fragments remain after such a procedure.

A probe tip that can be detachably coupled by an end-user to the probe body, e.g., to a probe body lumen of a lithotripsy device can allow for more efficient fragmentation of calculi such as by allowing the end-user to select and use a probe tip type that can be tailored to the calculi being treated. This can help better fragment a target calculus, which, in turn can help reduce the size of resulting particles to be evacuated via the probe. For example, a ceramic or composite type distal probe tip can be secured by an end-user to the probe body and aligned so that an evacuation passage through the tip and through the probe. This can allow for efficient breakdown of calculi and evacuation of calculi fragments. A particular probe tip can be easily detachable and replaceable by the end-user, such as with another probe tip of a different type or other characteristic, such as when desired by the end-user such as based on calculus type or any other reason.

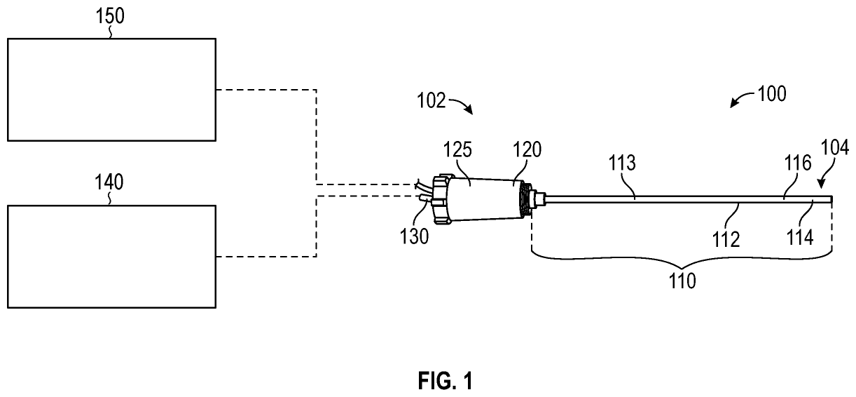

FIG. 1 illustrates a schematic diagram of an example of portions of an acoustically-transmissive calculi fracture and removal probe assembly 100 with an end-user interchangeable probe tip 114. The probe assembly 100 can include a proximal portion 102 and a distal portion 104. The probe assembly 100 can include a probe 110 with a probe body 112 and an end-user attachable or end-user detachable probe tip 114. The probe assembly 100 can also include or be coupled to one or more of an acoustic transducer 120, a handpiece 125, a evacuation passage 130, and a pressure source 140. The probe assembly 100 can be in communication with a generator 150.

The probe assembly 100 can include a lithotripsy device for treatment of calculi such as by fragmenting. The probe assembly 100 can provide lithotripsy treatment such as using ultrasonic or other acoustic energy, using low frequency solenoid-driven ballistic impact, or using any combination thereof, for fragmenting calculi or otherwise treating a physiological target. The probe assembly 100 can include a dual- or other multi-frequency device, such as can allow for pulsing of both sonic and ultrasonic waves for breakdown of calculi.

The probe 110 can be sized and shaped such as to allow for insertion into a patient, such as via an incision, such for treating calculi. The probe 110 can include an acoustically-transmissive probe for transfer of acoustic energy from a generator or acoustic transducer to a targeted calculus for fragmentation. The probe 110 can include a proximal portion 102, nearer the operator using the device, and a distal portion 104, nearer the site of treatment. The probe 110 can have a length of about 350 mm to about 600 mm, for example, depending on the specific probe type and end-user attachable or detachable distal probe tip used. The probe 110 can have a diameter of about 0.90 mm to about 3.80 mm, for example, depending on the specific probe type and end-user attachable or detachable distal probe tip used.

The probe 110 can include a probe body 112 extending between the proximal portion 102 and the distal portion 104, such as with a lumen 113 also extending between the proximal portion 102 and the distal portion 104. The probe body 112 can be sized and shaped for insertion into a patient, such as to reach a calculi for fragmentation. The probe body 112 can include or can be made of a metallic or composite metallic material. The probe body 112 can include one or more couplers or other attachment mechanisms for coupling with the probe tip 114. The probe body 112 can allow for the operator to manipulate the placement and actuation of the probe tip 114 on or near a target calculus.

The probe tip 114 can be selected by an end-user and attached to the probe body 112. The probe tip 114 can be sized, shaped, and arranged for breaking up, fragmenting, or fracturing, one or more targeted calculi. The probe tip 114 can be attached to the probe body 112. In some cases, the probe tip 114 can include a lumen 116. When the probe tip 114 is attached by an end-user to the probe body 112, the lumen 116 of the probe tip 114 can align with and extend from the lumen 113 of the probe body 112, such as to provide a contiguous irrigation and/or evacuation pathway. The probe tip 114 can have a desired morphology or other characteristic, such as a chiseled tip, a square tip, a tip with a large or small distally facing or periphery surface area, a varying topography, various morphology, or be of various materials, depending on the particular procedure to be performed or the particular target upon which the procedure is to be performed.

The acoustic transducer 120 can be actuatable for providing acoustic energy to the targeted calculi via the acoustically-transmissive probe 110. The acoustic transducer 120 can provide ultrasonic energy, sonic energy, or some combination thereof, such as to break down a targeted calculus, such as by fragmenting or dusting. In some cases, the acoustic transducer 120 can be configured for shock pulsing between various energy levels or energy types. This can include, for example, applying ultrasound energy with intermittent lower-frequency acoustic energy pulses or with intermittent ballistic mechanical energy doses. The acoustic transducer 120 can provide acoustic energy of varying waveforms or frequencies, depending on the particular operation. For example, the acoustic transducer 120 can be operated to select, adjust, or optimize the waveform for one or more portions of the procedure. The acoustic transducer 120 can be acoustically coupled to the acoustically-transmissive probe body 112, such as to provide acoustic energy down the length of the probe body 112 to the probe tip 114, which can be placed near or in contact with the targeted calculus. In an example, the acoustic transducer 120 can have a diameter of about 4 to about 6 cm, a length of about 15 to about 25 cm, and a weight of about 0.4 to about 1.0 kg, depending on the specific transducer used.

The handpiece 125 can be shaped and sized to allow for the end-user operator to grip and manipulate the probe assembly 100. In an example, the handpiece 125 can house all or a portion of the acoustic transducer 120. The handpiece 125 can include one or more buttons or other user interface means such as to allow the operator to control the probe assembly 100. For example, the handpiece 125 can include a dial for variable suction control in communication with the pressure source 140. In an example, the handpiece can include one or more buttons for applying ultrasonic, sonic, or other energy from the acoustic transducer 120, to apply to the targeted calculus for fragmentation. In some examples, the system can additionally or alternatively include a foot pedal or other auxiliary actuator, such as for controlling activation of the acoustic transducer 120.

The evacuation passage 130 can be fluidly connected to the lumen of the probe 110, such as to provide irrigation, suction, or both to the probe assembly 100. The evacuation passage 130 can extend outwards from the handpiece 125 towards a pressure source 140 or other pressure source. The pressure source 140 can provide an evacuation pressure down the length of the evacuation passage 130 to draw fragments of fractured calculi stones down the evacuation passage 130 away from the lumen of the probe 110. The evacuation passage 130 can additionally be irrigated as desired.

The generator 150 can be in electrical communication with the probe assembly 100, such as to provide electrical energy to the probe assembly 100 during use. The generator 150 can provide electrical energy to power the acoustic transducer 120 to generate ultrasound or other acoustic or ballistic energy such as for fragmenting a targeted calculus. In an example, the generator 150 can provide AC electrical energy of about 90 to about 264 volts (peak-to-peak). The electrical energy signal provided by the generator 150 can be changed (e.g., amplitude, frequency, pulse width, modulation, etc.) such as can depend on the particular treatment to be performed, and the desired parameters.

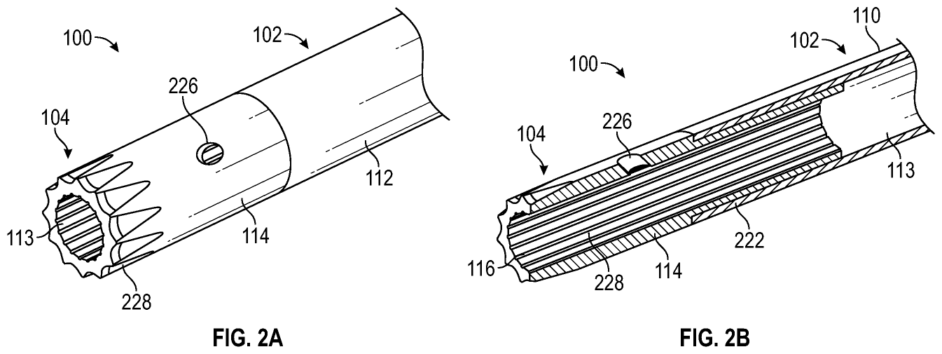

FIGS. 2A-2B illustrate an example of perspective views of portions of a probe assembly 100 with the user-interchangeable probe tip 114. The probe assembly 100 can include the proximal portion 102 and the distal portion 104. The probe assembly 100 can include the probe body 112 with a lumen 113. The probe assembly 100 can also include the probe tip 114 with an attachment feature 222, a lumen 116, one or more optional lateral openings 226, and one or more optional axial grooves 228. The probe assembly 100 can be used with an acoustic transducer and generator such as those discussed above with reference to FIG. 1 for the fragmentation of one or more targeted calculi stones.

The probe assembly 100 can be delivered to the operator with the probe tip 114 attached to the probe body 112, or can be delivered to the operator without the probe tip 114 being attached to the probe body 112 but instead with one or more probe tips 114 separately provided (e.g., as an auxiliary accessory or in a kit including various probe tips 114), which the operator can then (as the end-user) select a desired probe tip 114 and attach to the probe body 112 as desired. In some cases, a multitude of various probe tips 114 can be included for use with a single probe body 112. The multitude of probe tips 114 can be configured differently so that the end-user operator may switch a particular probe tip 114 in or out, such as depending on the particular procedure being performed or even specific to a particular target or portion of the procedure.

The probe body 112 can be shaped and sized to be end-user detachably connected to and work with the probe tip 114. The probe body 112 can extend from a proximal portion 102 to a distal portion 104. At the proximal portion 102, a handpiece and an acoustic transducer can be provided for operator use. At the distal portion 104, the probe body 112 can be end-user attachable to the probe tip 114.

The probe body 112 can be coupled to an acoustic energy source, such as the transducer discussed with reference to FIG. 1, for providing acoustic energy via the probe body to actuate the probe tip 114 to acoustically fracture the targeted calculus. The probe body 112 can include the lumen 113 longitudinally therethrough, such as to allow calculus fragments to be evacuated through the probe body 112 such as to a suction or evacuation tube or passage for collection or disposal.

The probe tip 114 can be user attachable and detachable from the probe body 112, such that an end-user operator of the probe assembly 100 can select or interchange various probe tips 114 as desired depending on the procedure being performed and the type of target calculus. The user-interchangeable probe tip 114 can be acoustically-transmissive to allow acoustic fracture, fragmentation, or dusting of target calculi. An acoustic impedance of the probe tip 114 can be matched to an acoustic impedance of the probe body 112, such as to transmit acoustic energy of a desired frequency across a boundary, junction, or joint between these two components, without substantial acoustic energy attenuation due to acoustic impedance mismatch between these two components. The probe tip 114 can be user-interchangeable without requiring a separate tool, such as explained herein, such that the probe tip 114 can be interchanged before or even during a procedure by the end-user operator, as opposed to during manufacturing of the probe assembly 100.

The probe tip 114 can include or can be made of a ceramic or composite material, such as zirconia, alumina, or a combination, and optionally doped with one or more of diamond, cubic zirconia, carbon nanotubes, tungsten, or combinations thereof. A lightweight material, such as a ceramic, can help avoid any significant increase the weight of the probe assembly 100 when the probe tip 114 is attached. This can yield a smaller mass overall that needs to be tuned or optimized to the transducer or the probe body 112 for effective transfer of acoustic energy to the targeted calculus. The material of the probe tip 114 can be acoustically transmissive, such as at the ultrasound or acoustic frequency being used for treatment.

The probe tip 114 can be any number of interchangeable probe tips of varying morphology. For example, the probe tip 114 can be chosen from a variety of interchangeable probe tips that allow for varying contact pressure depending on the targeted calculus type. For example, a probe tip with a broad tip can be used for softer calculus, and a probe tip with a sharp tip can be used for a harder calculus. For example, a square ended probe tip, with a larger amount of surface area for contact with the calculus, can be used for a softer calculus. In contrast, a chiseled, serrated, segmented, or cupped end probe tip with a more targeted, smaller surface area for contact with a calculus can be used to target and breakup a harder calculus. In some cases, a dual purpose probe tip, such as with a beveled edge, or an angled edge (such as about 45 degrees), can be used for a probe tip with both needle like properties for targeting harder calculus, and grinding type properties for targeting softer calculus.

The probe tip 114 can be secured and attached to the probe body 112 by the end-user operator, such as through one or more couplers or attachments, such as can provide mating or other engagement therebetween. For example, a coupler on the probe tip 114 can include one or more toolless attachments and/or interlocks that are user-manipulatable with respect to the probe body 112 for engagement, attachment, and/or locking thereto, and for disengagement or detachment therefrom. The attachment feature 222 can include threads or other protrusions or grooves or shapes to engage corresponding complementary features of the probe body 112, such as to lock or otherwise help mechanically secure the probe tip 114 to the probe body 112. In an example, the attachment feature 222 can include threads on the interior diameter of the probe body 112 and corresponding screw features on the probe tip 114. In another example, the attachment feature 222 can include a right turn slot in the probe body 112 corresponding to a hub post on the probe tip 114, such as a post that clicks into the slot with a quarter turn. In some cases, the outer diameter of the probe 110 could be swaged to allow for better diametral fit. In an example, the attachment feature 222 can include an inward roll form on a distal end of the probe 110. In this case, a relief groove could be present in the device to interact with the roll form. Additional relief cuts can allow for improved diametral fit. In some cases, inward spring hooks could be used. In another example, the attachment feature 222 can include an outward roll form, with a corresponding inner groove. In some cases, external spring hooks can be used. In some cases, the attachment feature 222 can additionally or alternatively include an adhesive.

For example, the probe body 112 can include the lumen 113 into which a reduced outer diameter interference-fit attachment feature 222 of probe tip 114 can be inserted. The interference fit attachment feature 222 of the probe tip 114 can be inserted such that the probe tip 114 is securely engaged with the probe body 112, such as when a matching outer diameter portion of the probe tip 114 is placed into abutment of a distal end of the probe body 112 with the interference-fit attachment feature 222 inserted into the lumen of the probe body 112. The interference fit can allow for attachment and retention without locking.

In an example, the attachment feature 222 can be a locking feature that allows for attachment, engagement, and locking of the probe tip 114 to the probe body 112. Such locking engagement can include a quarter-turn interlock or similar retention system in which the probe tip 114 is inserted by the end-user into the probe body 112, and then rotated by a quarter-turn or other specified amount to lock the probe tip 114 into the probe body.

In an example, the attachment feature 222 can be a snap fit. A snap fit can include pushing one or more interlocking components, such as protrusions, grooves, or other geometric shapes on the probe tip 114 into corresponding features on the probe body 112. Such a snap fit can include cantilever, torsional, annular, or combination snap fits, depending on the shape and size of the probe body 112 and the probe tip 114. A snap fit can be used to both attached and interlock the probe body 112 and the probe tip 114.

In an example, the attachment feature 222 can be a threaded feature. For example, threads can be in the inner wall of the lumen 113 of the probe bodyl12, with corresponding threads on the outer wall of the probe tip 114. In other examples, the threads can be on the outside of the probe body 112. The threads can allow for attachment and securing of the probe tip 114 to the probe body 112.

The probe tip 114 can include the longitudinal lumen 116 that, when the probe tip 114 is secured to the probe body 112, is aligned with the lumen 113 of the probe body 112, such as to allow fragmented calculi portions to be removed through a suction or evacuation passage defined by the lumens 113, 116 and optionally a separate evacuation passage.

The probe tip 114 can include one or more lateral openings 226, such as openings into the lumen 113 from a nearby lateral region outside of the probe tip 114. The lateral openings 226 can allow for influx of a fluid into the lumen 116 of the probe tip 114, such as to help flush fragments of calculi in the lumen 116. The lateral openings 226 can help promote, increase, or maximize fluid flow in one or both of the lumens 113, 116, such as can help transport fragments of calculi when the distal end opening of the probe tip 114 into the lumen 116 becomes partially or fully blocked. The location of the lateral openings 226 on the replaceable probe tip 114 can help inhibit or prevent stress risers and fracture points that could otherwise occur if the lateral openings 226 were located in the probe body 112, which is vibrated during operation, and which may be intended to be more durable and reusable than the probe tip 114, which is capable of being discarded or replaced by the end-user, as explained herein.

The probe tip 114 can additionally include one or more grooves or channels, such as one or more axial grooves 228. The axial grooves 228 within the lumen 116 of the probe tip 114 can help promote flow of fluid within the lumen 116, such as when the opening of the probe tip 114 is partially blocked. The axial grooves 228 can additionally allow for fluid flow when the lumen 113 of the probe body 112 and the lumen 116 of the probe tip 114 are occluded. The axial grooves 228 can extend along the probe tip 114, or extend further into and along the probe body 112, as desired.

An end-user operator, such as a surgeon, can select a particular probe tip 114 (such as from a kit of available multiple probe tips 114) that is best suited to a particular procedure or to a particular calculus or group of calculi to be treated. For example, the particular probe tip 114 can be chosen based on the material of one or both of the probe tip or probe body, based on the morphology of the probe tip, based on the acoustic impedance of the probe tip, based on a desired acoustic energy range for the procedure, the surface area of the probe tip itself, other dimensions of the probe tip, or one or more combinations of those parameters.

These types of probe-tip selection parameters can be correlated to one or both of the procedure being done and a type or other characteristic of a calculus to be fragmented. For example, a harder calculus may be more efficiently fragmented with a chiseled tip profile, which can allow for more specific directionality or regions of cutting or fragmenting. A softer calculi stone may be more efficiently fragmented with a squared tip profile, or a tip profile with a larger surface area. The probe tip 114 can help provide acoustic impedance matching, e.g., to the probe body 112 and the acoustic transducer, to the target calculus, or both. This can help provide more efficient acoustic energy transfer from the probe body 112 to the target calculus, and such additional flexibility can help deliver acoustic energy to the target calculus in a desired manner, such as at or near a characteristic resonance frequency of the target calculus, such as by properly selecting a particular probe tip 114 from a kit or set of available probe tips 114.

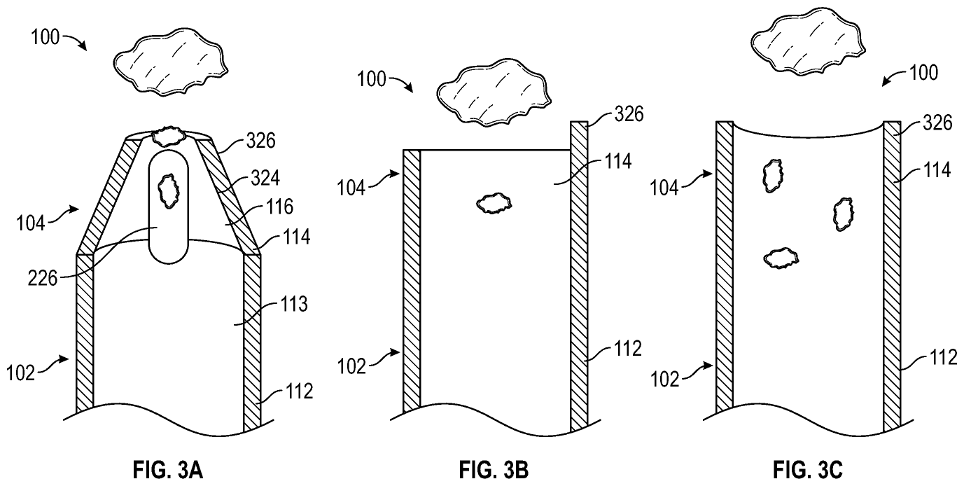

FIGS. 3A-3C illustrate schematic diagrams of a probe assembly 100 with an interchangeable probe tip 114 having one or more tongs 326. The probe assembly 100 can have a proximal portion 102 and a distal portion 104. The probe assembly 100 can include a probe body 112 and a probe tip 114. The probe body 112 can include a lumen 113. The probe tip 114 can have an inner diameter 324 defining a lumen 116, along which tongs 326 can rest. The probe tip 114 can additionally include one or more lateral openings 226. The probe tip 114 can be detachable from the probe body 112 and can be attached such that the lumen 113 and the lumen 116 are aligned.

In one approach, a probe tip can have an inner diameter or similar dimension across a lumen wall and in which both the inner diameter and the lumen wall shape is consistent through the length of the probe tip. In another approach, such as shown for the probe tip 114, the inner diameter 324 of the lumen can gradually increase such as from the distal end opening probe tip 114 toward the probe body 112, such as can define the lumen 116 as having a longitudinally tapered inner diameter. In this approach, the longitudinally tapered inner diameter 324 can help allow for better movement of calculi fragments through the lumen 116 away from the probe tip 114 towards the lumen 113 of the probe body 112.

The probe tip 114 can have a laterally smaller tip hole at the distal portion 104, compared to other probe tips, including compared to one or more other probe tips that may be included therewith in a kit or set. A smaller hole at the probe tip 114 can be drawn down from a larger size at a more proximal location at various angle and profiles.

One or more tongs 326 can extend outwardly from the probe tip 114. The tongs 326 can allow for drawn down, such as capture and movement of calculi fragments down and into the probe 110, during operation, such that calculi fragments move towards the lumen 113 of the probe body 112 and towards an evacuation or evacuation passage. The tongs 326 can include multiple tongs, as desired, at varying angles, depending on the type of calculi stone being treated and the size and types of fragments expected. The tongs 326 can extend distally outward from the probe tip 114 so as to create a larger space and allow movement of stones or stone fragments into the tongs 326. The tongs 326 can be straight or pointed inwards at varying degrees. The lateral openings 226, cut into the probe tip 114, can allow for suction and removal of stone fragments.

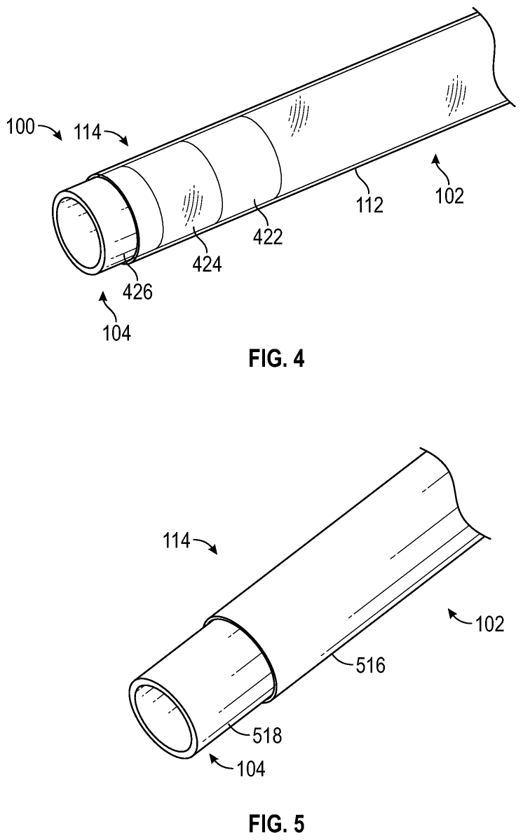

FIG. 4 illustrates a schematic diagram of an end-user interchangeable probe tip in a probe assembly 100. The probe assembly 100 can have a proximal portion 102 and a distal portion 104. The probe assembly 100 can include a probe body 112 and probe tip 114 with portions 422, 424, 426. In probe assembly 100, the distal portion 104 can host the probe tip 114, which can be adjustable in length at the end of the probe body 112.

For example, the probe tip 114 can include three or more ring like portions 422, 424, 426. The operator can adjust the number of portions 422, 424, 426, that are distally extended relative the probe body 112 for use. The probe tip 114 can be slidable relative to the probe body 112, in a longitudinal direction between the proximal portion 102 and the distal portion 104. The probe tip portions 422, 424, 426 can be slidable relative each other, depending on the desired probe tip shape, size, and length, for treatment of targeted calculi. The probe tip portions 422, 424, and 426 can nest into each other. They can be connected to each other, for example, by an interference fit, a snap fit, a thread, a quarter turn interlock, or other mechanisms that allow the tip portions 422, 424, 426 to be laterally moved outwards from each other.

In use, the first portion 422 can be used for fracture of targeted calculi stone. The first portion 422 can be laterally extended in a distal direction from the probe body 112. The second portion 424 and the third portion 426 can be nested within the first portion 422 at the beginning of the operation. If the first portion 422 is damaged during the operation, or if the operator desires to reach further distally with the assembly 100, the second portion 424 can be pushed out from within the first portion 422 for use. When the second portion 424 is extended, it can still be attached to the first portion 422, effectively lengthening the probe assembly 100. Similarly, the third portion 426 can be pushed out from within the second portion 424 if the operator no longer wishes to use the second portion 424. The portions 422, 424, and 426, can stay attached to the assembly 100 throughout this process. The portions 422, 424, and 426, do not detach during operation.

The portions 422, 424, 426, can be actuatable by one or more triggers on a handpiece of the assembly, such as one or more buttons, levers, or a roller wheel. In the case of the roller wheel, such a wheel could be coupled to the portions 422, 424, 426, and actuation with a finger or thumb could cause the portions 422, 424, 426, to slidably move along the assembly 100, such as in a distal direction towards the distal portion 104, which a new portions is desired. In some cases, the portions 422, 424, 426, can be actuated through a mechanical mechanism, such as one or more springs integrated into the probe body 112. In this case, the spring can be compressed or released to push or pull the portions 422, 424, and 426, along an axis of the probe 110. In some cases, the mechanism can be a hydraulic, electromagnetic, or an air piston actuator to move the portions 422, 424, 426 in or out of the probe body 112.

FIG. 5 illustrates a schematic diagram of an example of portions of an interchangeable probe tip in a probe assembly 100. The probe assembly 100 can have a proximal portion 102 and a distal portion 104. The probe assembly 100 can include the probe tip 114 having a sheath piece 516 and a tip piece 518. Here, the sheath piece 516 can act as a sheath encircling the tip piece 518. The probe tip 114 tip piece 518 can be laterally moveable along the sheath piece 516, so that an end-user can adjust the distal positioning of the tip piece 518 of the probe tip 114 in and out of the sheath piece 516 In probe assembly 100, the distal portion 104 can host the sheath piece 516, which is adjustable in length at the end of the sheath piece 516. The sheath piece 516 can be positioned in and end-user attachable to one or more additional pieces of the probe, such as the probe body 112. The probe tip 114 can be coupled to the probe body 112 and allow for slidable movement of the tip piece 518 along the sheath piece 516 such that an operator can move the tip piece 518 distally along the sheath piece 516 while the probe body 112 stays constant. An operator can adjust the position of the tip piece 518 relative the sheath piece 516 as desired for the particular operation. The probe tip 114 tip piece 518 can come into contact with the targeted calculi stone. The end-user can move the probe tip 114 tip piece 518 relative the sheath piece 516 with a switch, dial, or other trigger mechanically coupled to the probe tip 114. The mechanical trigger can be integrated into the handpiece 125 for easy access, and to allow articulation and lateral movement of the probe tip 114 .

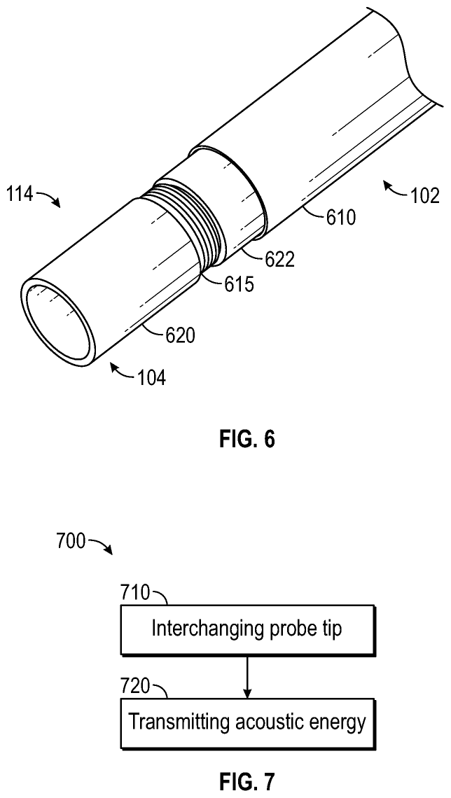

FIG. 6 illustrates a schematic diagram of an interchangeable probe tip in a probe assembly 100, similar to the probe assembly discussed with reference to FIG. 5 above. The probe assembly 100 can have a proximal portion 102 and a distal portion 104. The probe assembly 100 can include the probe tip 114 with a probe delivery sheath 610, an inner rotation tube 615, and a probe ring 620 with threaded portion a 622. In probe assembly 100, the distal portion 104 can host the inner rotation tube 615, probe ring 620, and threaded portion 622. In the probe tip 114, the inner rotation tube 615, the probe ring 620, the threaded portion 622 are laterally moveable relative the probe delivery sheath 610.

The probe ring 620 can be slidable within the probe delivery sheath 610. The probe ring 620 can contain a threaded portion 622 distal the probe delivery sheath 610 and connected by the inner rotation tube 615, which can allow for attachment of the probe tip 114 to the probe body 112. In this way, the probe tip 114 can be interchanged with the assembly, and the assembly can allow for distal and proximal articulation and positioning of the same.

FIG. 7 illustrates a flow chart showing a method 700 of treating a calculi stone. Method 700 can include steps 710 and 720. The process can optionally include selecting a probe tip from a set of available probe tips, or from a kit containing the various probe tips. In this case, the kit can, for example, include probe tips of varying surface morphology or materials, or other variants as discussed above. The kit can, for example, include probe tips that are labeled for operator use, such as labeled for hard calculi, soft calculi, large calculi, small calculi, and other parameters correlating to the type of calculi mass that is being treated.

Step 710 can include interchanging a probe tip with a probe body. The probe tip can be interchanged with the probe body by a user, such as a surgeon or other operator, during, or before a procedure. The probe tip can be interchanged without requiring additional tools or manufacturing techniques.

Step 720 can include transmitting acoustic energy via the probe body and the probe tip to a calculi to at least partially fracture the calculi. The energy provided via the probe tip to the calculi can fragment, dust, or otherwise fracture the targeted calculi stone.

The method 700 can allow for customization of probe tips, such as customized manufacturing of probe tips, based on a specific patient's needs and the procedure to be performed. For example, diagnostic tools can be used to identify the size and type of stone needing to be removed, and 3D printing or high-speed machining can fabricate the corresponding desired probe.

For example, the probe tip can be selected for step 710 based on one or more parameters of the targeted calculi stone, such as stone size, stone density, stone type, or one or more combinations thereof. In some cases, more than one probe can be used during the procedure, such that interchanging the probe tip can include exchanging for an alternative probe tip during a medical procedure based on a parameter of the calculi.

### Various Notes & Examples

Each of these non-limiting examples can stand on its own or can be combined in various permutations or combinations with one or more of the other examples.

Example 1 can include a device for acoustic calculi fracture. The device can include an acoustically-transmissive elongated probe body extending between a distal portion and a proximal portion, the probe body having a lumen longitudinally therethrough, and an acoustically-transmissive probe tip selectively user-interchangeable with the probe body.

Example 2 can include Examples 1, wherein the acoustically-transmissive probe tip is selectively user-interchangeable with the probe body without requiring a separate tool.

Example 3 can include any of Examples 1-2, further comprising an acoustic energy source actuatable for providing acoustic energy via the probe body such that the probe tip is actuated for acoustic fracture of one or more calculi via the probe tip.

Example 4 can include any of Examples 1-3, wherein the probe-tip includes a toolless interlock user-manipulatable with respect to the probe body to interchange the probe tip with the probe body.

Example 5 can include any of Examples 1-4, wherein the probe tip comprises a ceramic or a composite ceramic material.

Example 6 can include any of Examples 1-5, wherein the probe tip includes a longitudinal lumen configured to align with the lumen of the probe body.

Example 7 can include any of Examples 1-6, wherein the probe tip comprises a lateral opening from the longitudinal lumen to a surrounding lateral region outside of the probe tip.

Example 8 can include any of Examples 1-7, wherein the lateral opening is configured to allow influx of fluid into the lumen of the probe body via at least a portion of the longitudinal lumen of the probe tip.

Example 9 can include any of Examples 1-8, wherein the probe tip further comprises one or more axial grooves.

Example 10 can include any of Examples 1-9, wherein a distance between a distal end of the probe tip and a distal end of the probe body is at least one of user-adjustable or user-selectable via user-interchanging of the probe tip.

Example 11 can include any of Examples 1-10, wherein the distance between the distal end of the probe tip and the distal end of the probe body is user-adjustable and the probe body is slidable relative to the probe body along a longitudinal axis of the probe body.

Example 12 can include a kit for a calculi fracture device, comprising a plurality of different acoustically-transmissive probe tips that are selectively user-interchangeable with a probe body of a lithotripsy device without requiring a separate tool.

Example 13 can include Example 12, wherein at least one of the probe tips further comprises a locking feature for securing the probe tip to the probe body.

Example 14 can include any of Examples 12-13, further comprising an acoustic energy source attached to the probe body for providing acoustic energy via the probe body.

Example 15 can include any of Examples 12-14, wherein the different probe tips are different in at least one of the following characteristics: material; tip morphology; acoustic impedance; tip surface area; or tip dimension; or one or more combinations thereof

Example 16 can include any of Examples 12-15, wherein the probe tips are attachable to the probe body with one or more attachment mechanisms.

Example 17 can include a method of fracturing calculi, comprising: interchanging probe tip with a probe body by a user; and transmitting acoustic energy via the probe body and the probe tip to a calculi to at least partially fracture the calculi.

Example 18 can include Example 17, further comprising selecting the probe tip based on one or more parameters of the calculi.

Example 19 can include any of Examples 17-18, wherein the one or more parameters comprise stone size, stone density, stone type, or one or more combinations thereof.

Example 20 can include any of Examples 17-19, further comprising interchanging the probe tip with an alternative probe tip during a medical procedure based on a parameter of the calculi.

Example 21 can include any of Examples 17-20, wherein interchanging the probe tip with the probe body by the user comprises interchanging the probe tip without requiring an additional tool.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device for acoustic calculi fracture, comprising:
an acoustically-transmissive elongated probe body extending between a distal portion and a proximal portion, the probe body having a lumen longitudinally therethrough; and
an acoustically-transmissive probe tip selectively user-interchangeable with the probe body.

2. The device of claim 1, wherein the acoustically-transmissive probe tip is selectively user-interchangeable with the probe body without requiring a separate tool.

3. The device of claim 1, further comprising an acoustic energy source actuatable for providing acoustic energy via the probe body such that the probe tip is actuated for acoustic fracture of one or more calculi via the probe tip.

4. The device of claim 1, wherein the probe-tip includes a toolless interlock user-manipulatable with respect to the probe body to interchange the probe tip with the probe body.

5. The device of claim 1, wherein the probe tip comprises a ceramic or a composite ceramic material.

6. The device of claim 1, wherein the probe tip includes a longitudinal lumen configured to align with the lumen of the probe body.

7. The device of claim 6, wherein the probe tip comprises a lateral opening from the longitudinal lumen to a surrounding lateral region outside of the probe tip.

8. The device of claim 7, wherein the lateral opening is configured to allow influx of fluid into the lumen of the probe body via at least a portion of the longitudinal lumen of the probe tip.

9. The device of claim 1, wherein the probe tip further comprises one or more axial grooves.

10. The device of claim 1, wherein a distance between a distal end of the probe tip and a distal end of the probe body is at least one of user-adjustable or user-selectable via user-interchanging of the probe tip.

11. The device of claim 10, wherein the distance between the distal end of the probe tip and the distal end of the probe body is user-adjustable and the probe body is slidable relative to the probe body along a longitudinal axis of the probe body.

12. A kit for a calculi fracture device, comprising:
a plurality of different acoustically-transmissive probe tips that are selectively user-interchangeable with a probe body of a lithotripsy device without requiring a separate tool.

13. The kit of claim 12, wherein at least one of the probe tips further comprises a locking feature for securing the probe tip to the probe body.

14. The kit of claim 12, further comprising an acoustic energy source attached to the probe body for providing acoustic energy via the probe body.

15. The kit of claim 12, wherein the different probe tips are different in at least one of the following characteristics:
material;
tip morphology;
acoustic impedance;
tip surface area; or
tip dimension;
or one or more combinations thereof.

16. The kit of claim 12, wherein the probe tips are attachable to the probe body with one or more attachment mechanisms.

17. A method of fracturing calculi, comprising:
interchanging a probe tip with a probe body by a user; and
transmitting acoustic energy via the probe body and the probe tip to a calculi to at least partially fracture the calculi.

18. The method of claim 17, further comprising selecting the probe tip based on one or more parameters of the calculi.

19. The method of claim 18, wherein the one or more parameters comprise stone size, stone density, stone type, or one or more combinations thereof.

20. The method of claim 17, further comprising interchanging the probe tip with an alternative probe tip during a medical procedure based on a parameter of the calculi.
